(19) United States
(12) Patent Application Publication
(10) Pub. No.: US 2008/0208049 A1
(43) Pub. Date: Aug. 28, 2008

(54) ULTRASONIC DIAGNOSTIC APPARATUS AND ULTRASONIC DIAGNOSTIC APPARATUS SYSTEM

(76) Inventors: Hiroshi Hashimoto, Tokyo (JP); Takao Jibiki, Tokyo (JP)

Correspondence Address: Patrick W. Rasche;Armstrong Teasdale LLP, Suite 2600, One Metropolitan Square, St. Louis, MO, 63102, US

(21) Appl. No.: 12/034,430

(22) Filed: Feb. 20, 2008

(30) Foreign Application Priority Data
Feb. 23, 2007 (JP) 2007-043139

(51) Int. Cl. A61B 8/14

(52) U.S. Cl. 600/437

(57) ABSTRACT

An ultrasonic diagnostic apparatus includes a contrast timer starting time measurement by a start signal and an external input device for externally inputting said start signal.

### CROSS REFERENCE TO RELATED APPLICATIONS

This application claims the benefit of Japanese Patent Application No. 2007-0431139 filed Feb. 23, 2007.

### BACKGROUND OF THE INVENTION

The field of the present invention relates to an ultrasonic diagnostic apparatus and an ultrasonic diagnostic apparatus system. More specifically, the present invention relates to an ultrasonic diagnostic apparatus and an ultrasonic diagnostic apparatus system that can synchronize contrast timers of a plurality of ultrasonic diagnostic apparatuses with each other.

There is an ultrasonic diagnostic apparatus having a stopwatch function which starts time measurement by start operation of an operator, stops time measurement by a freeze instruction of the operator, and displays an elapsed time from start on a screen. The function is called a contrast timer.

Using the stopwatch function, start operation is performed when a contrast agent is injected into a subject, and then, operation of the freeze instruction is performed at a suitable point in time. An elapsed time from injection of the contrast agent to photographing of a freeze ultrasonic image can be checked on a screen (For instance, see Patent Document 1).

Patent Document 1. Japanese Unexamined Patent Publication No. 148015/2004.

There may be cases where plural sites (for instance, a liver and a thyroid gland) of a subject with a contrast agent injected thereinto are desired to be photographed by a plurality of ultrasonic diagnostic apparatuses.

The prior art ultrasonic diagnostic apparatus is required to perform start operation of the contrast timers of the respective ultrasonic diagnostic apparatuses. The operation can be troublesome. Simultaneous start cannot be accurate.

### SUMMARY OF THE INVENTION

It is desirable that the problem described previously is solved.

In a first aspect, the invention provides an ultrasonic diagnostic apparatus including a contrast timer starting time measurement by a start signal, and an external input device for externally inputting the start signal.

In the ultrasonic diagnostic apparatus according to the first aspect, the start signal is externally inputted to a plurality of ultrasonic diagnostic apparatuses at the same time. The contrast timers of the plurality of ultrasonic diagnostic apparatuses can be started at the same time.

In a second aspect, the invention provides an ultrasonic diagnostic apparatus including a contrast timer starting time measurement by a start signal, an operating device for use by an operator to perform operation for producing the start signal, and an external output device for externally outputting the start signal.

In the ultrasonic diagnostic apparatus according to the second aspect, the start signal is transmitted to the ultrasonic diagnostic apparatus according to the first aspect. The contrast timer of the ultrasonic diagnostic apparatus according to the first aspect and the contrast timer of the ultrasonic diagnostic apparatus according to the second aspect can be started at the same time.

In a third aspect, the invention provides an ultrasonic diagnostic apparatus including a contrast timer starting time measurement by a start signal, an operating device for use by an operator to perform operation for producing the start signal, an external output device for externally outputting the start signal, and an external input device for externally inputting the start signal.

In the ultrasonic diagnostic apparatus according to the third aspect, the start signal is externally inputted to a plurality of ultrasonic diagnostic apparatuses at the same time. The contrast timers of the plurality of ultrasonic diagnostic apparatuses can be started at the same time. The start signal is transmitted from one ultrasonic diagnostic apparatus to another ultrasonic diagnostic apparatus. Accordingly, the contrast timers of the plurality of ultrasonic diagnostic apparatuses can be started at the same time.

In a fourth aspect, the invention provides the ultrasonic diagnostic apparatus according to the third aspect further including an external input inhibiting device for inhibiting an external input of the start signal for a predetermined time after the start signal is produced by operation of the operating device.

When the ultrasonic diagnostic apparatus according to the fourth aspect is connected in a loop, the start signal can be transmitted from any ultrasonic diagnostic apparatus to another ultrasonic diagnostic apparatus. Since the external input of the start signal is inhibited for a predetermined time after the start signal is produced by operation of the operating device, the start signal can be prevented from being circulated in the loop.

In a fifth aspect, the invention provides an ultrasonic diagnostic apparatus system including an ultrasonic diagnostic apparatus according to the first aspect, an ultrasonic diagnostic apparatus according to the second aspect, and a communicating device for connecting the external input device of the ultrasonic diagnostic apparatus according to the first aspect and the external output device of the ultrasonic diagnostic apparatus according to the second aspect.

In the ultrasonic diagnostic apparatus system according to the fifth aspect, the start signal is transmitted from the ultrasonic diagnostic apparatus according to the second aspect to the ultrasonic diagnostic apparatus according to the first aspect. Accordingly, the contrast timers of both the ultrasonic diagnostic apparatuses can be started at the same time.

In a sixth aspect, the invention provides an ultrasonic diagnostic apparatus system including a first ultrasonic diagnostic apparatus according to the fourth aspect, a second ultrasonic diagnostic apparatus according to the fourth aspect, a first communicating device for connecting the external output device of the first ultrasonic diagnostic apparatus and the external input device of the second ultrasonic diagnostic apparatus, and a second communicating device for connecting the external input device of the first ultrasonic diagnostic apparatus and the external output device of the second ultrasonic diagnostic apparatus.

In the ultrasonic diagnostic apparatus system according to the sixth aspect, the start signal is transmitted from the first ultrasonic diagnostic apparatus to the second ultrasonic diagnostic apparatus, whereby the contrast timers of both the ultrasonic diagnostic apparatuses can be started at the same time. The start signal is transmitted from the second ultrasonic diagnostic apparatus to the first ultrasonic diagnostic apparatus, whereby the contrast timers of both the ultrasonic diagnostic apparatuses can be started at the same time.

In a seventh aspect, the invention provides an ultrasonic diagnostic apparatus system including a first ultrasonic diagnostic apparatus according to the first aspect, a second ultrasonic diagnostic apparatus according to the first aspect, an instruction device having operating device for use by an operator to perform operation for producing a start signal and external output device for externally outputting a plurality of the start signals, and a communicating device for connecting the external input device of the first ultrasonic diagnostic apparatus, the external input device of the second ultrasonic diagnostic apparatus, and the external output device of the instruction device.

In the ultrasonic diagnostic apparatus system according to the seventh aspect, the start signal is transmitted from the instruction device to the first and second ultrasonic diagnostic apparatuses. Accordingly, the contrast timers of both the ultrasonic diagnostic apparatuses can be started at the same time.

In an eighth aspect, the invention provides an ultrasonic diagnostic apparatus system including a first ultrasonic diagnostic apparatus according to the third or fourth aspect, a second ultrasonic diagnostic apparatus according to the third or fourth aspect, and a communicating device for connecting the external output device of the first ultrasonic diagnostic apparatus and the external input device of the second ultrasonic diagnostic apparatus.

In the ultrasonic diagnostic apparatus system according to the eighth aspect, the start signal is transmitted from the first ultrasonic diagnostic apparatus to the second ultrasonic diagnostic apparatus. Accordingly, the contrast timers of both the ultrasonic diagnostic apparatuses can be started at the same time.

In a ninth aspect, the invention provides an ultrasonic diagnostic apparatus system including, when n=1, 2, . . . , N−1, N≧3, the first to N-th ultrasonic diagnostic apparatuses according to the third or fourth aspect, and the n-th communicating device for connecting external output device of the n-th ultrasonic diagnostic apparatus and the external input device of the (n+1)-th ultrasonic diagnostic apparatus.

In the ultrasonic diagnostic apparatus system according to the ninth aspect, the start signal is outputted from the first ultrasonic diagnostic apparatus. Accordingly, the contrast timers of three or more ultrasonic diagnostic apparatuses can be started at the same time.

In a tenth aspect, the invention provides an ultrasonic diagnostic apparatus system including an ultrasonic diagnostic apparatus according to the second aspect, an ultrasonic diagnostic apparatus according to the third aspect, and communicating device for connecting external output device of the ultrasonic diagnostic apparatus according to the second aspect and external input device of the ultrasonic diagnostic apparatus according to the third aspect.

In the ultrasonic diagnostic apparatus system according to the tenth aspect, the start signal is transmitted from the ultrasonic diagnostic apparatus according to the second aspect to the ultrasonic diagnostic apparatus according to the third aspect. Accordingly, the contrast timers of both the ultrasonic diagnostic apparatuses can be started at the same time.

In an eleventh aspect, the invention provides an ultrasonic diagnostic apparatus system including an ultrasonic diagnostic apparatus according to the second aspect, an ultrasonic diagnostic apparatus according to the fourth aspect, and communicating device for connecting the external output device of the ultrasonic diagnostic apparatus according to the second aspect and external input device of the ultrasonic diagnostic apparatus according to the fourth aspect.

In the ultrasonic diagnostic apparatus system according to the eleventh aspect, the start signal is transmitted from the ultrasonic diagnostic apparatus according to the second aspect to the ultrasonic diagnostic apparatus according to the fourth aspect. Accordingly, the contrast timers of both the ultrasonic diagnostic apparatuses can be started at the same time.

In a twelfth aspect, the invention provides an ultrasonic diagnostic apparatus system including a first ultrasonic diagnostic apparatus according to the third or fourth aspect, a second ultrasonic diagnostic apparatus according to the third or fourth aspect, an instruction device having an operating device for use by an operator to perform operation for producing a start signal and an external output device for externally outputting the start signal, a first communicating device for connecting the external input device of the first ultrasonic diagnostic apparatus and the external output device of the instruction device, and a second communicating device for connecting the external output device of the first ultrasonic diagnostic apparatus and the external input device of the second ultrasonic diagnostic apparatus.

In the ultrasonic diagnostic apparatus system according to the twelfth aspect, the start signal is transmitted from the instruction device to the first ultrasonic diagnostic apparatus. Accordingly, the contrast timers of the first and second ultrasonic diagnostic apparatuses can be started at the same time.

In a thirteenth aspect, the invention provides an ultrasonic diagnostic apparatus system including, when n=1, 2, . . . , N−1, N≧3, first to N-th ultrasonic diagnostic apparatuses according to the fourth aspect, the n-th communicating device for connecting the external output device of the n-th ultrasonic diagnostic apparatus and the external input device of the (n+1)-th ultrasonic diagnostic apparatus, and the N-th communicating device for connecting the external output device of the N-th ultrasonic diagnostic apparatus and the external input device of the first ultrasonic diagnostic apparatus.

In the ultrasonic diagnostic apparatus system according to the thirteenth aspect, three or more ultrasonic diagnostic apparatuses are connected in a loop. Accordingly, the start signal can be transmitted from any ultrasonic diagnostic apparatus to another ultrasonic diagnostic apparatus. The external input of the start signal is inhibited for a predetermined time after the start signal is produced by operation of the operating device. Accordingly, the start signal can be prevented from being circulated in the loop.

In a fourteenth aspect, the invention provides an ultrasonic diagnostic apparatus including a contrast timer starting time measurement by a start signal and stopping time measurement by a stop signal, an operating device for use by an operator to perform operation for producing the start signal and the stop signal, an external output device for externally outputting the start signal and the stop signal, and an external input device for externally inputting the start signal and the stop signal.

In the ultrasonic diagnostic apparatus according to the fourteenth aspect, the start signal or the stop signal is externally inputted to a plurality of ultrasonic diagnostic apparatuses at the same time. Accordingly, the contrast timers of the plurality of ultrasonic diagnostic apparatuses can be started or stopped at the same time. The start signal or the stop signal is transmitted from one ultrasonic diagnostic apparatus to another ultrasonic diagnostic apparatus. Accordingly, the contrast timers of the plurality of ultrasonic diagnostic apparatuses can be started or stopped at the same time.

In a fifteenth aspect, the invention provides an ultrasonic diagnostic apparatus system including a first ultrasonic diagnostic apparatus according to the fourteenth aspect, a second ultrasonic diagnostic apparatus according to the fourteenth aspect, and a communicating device for connecting the external output device of the first ultrasonic diagnostic apparatus and the external input device of the second ultrasonic diagnostic apparatus.

In the ultrasonic diagnostic apparatus system according to the fifteenth aspect, the start signal or the stop signal is transmitted from the first ultrasonic diagnostic apparatus to the second ultrasonic diagnostic apparatus. Accordingly, the contrast timers of both the ultrasonic diagnostic apparatuses can be started or stopped at the same time.

In a sixteenth aspect, the invention provides an ultrasonic diagnostic apparatus system including, when n=1, 2, . . . , N−1, N≧3, the first to N-th ultrasonic diagnostic apparatuses according to the first aspect, and the n-th communicating device for connecting the external output device of the n-th ultrasonic diagnostic apparatus and the external input device of the (n+1)-th ultrasonic diagnostic apparatus.

In the ultrasonic diagnostic apparatus system according to the sixteenth aspect, the start signal or the stop signal is outputted from the first ultrasonic diagnostic apparatus. Accordingly, the contrast timers of three or more ultrasonic diagnostic apparatuses can be started or stopped at the same time.

According to the ultrasonic diagnostic apparatus and the ultrasonic diagnostic apparatus system of the invention, contrast timers of a plurality of ultrasonic diagnostic apparatuses can be easily and accurately synthesized with each other.

The ultrasonic diagnostic apparatus and the ultrasonic diagnostic apparatus system of the invention can be used for photographing plural sites (for instance, a liver and a thyroid gland) of a subject with a contrast agent injected thereinto by a plurality of ultrasonic diagnostic apparatuses.

Further objects and advantages of the present invention will be apparent from the following description of the preferred embodiments of the invention as illustrated in the accompanying drawings.

### BRIEF DESCRIPTION OF THE DRAWINGS

FIG. 1 is a configuration explanatory view showing an ultrasonic diagnostic apparatus according to Embodiment 1.

FIG. 2 is a configuration explanatory view showing an ultrasonic diagnostic apparatus according to Embodiment 2.

FIG. 3 is a configuration explanatory view showing an ultrasonic diagnostic apparatus according to Embodiment 3.

FIG. 4 is a configuration explanatory view showing an ultrasonic diagnostic apparatus system according to Embodiment 4.

FIG. 5 is a configuration explanatory view showing an ultrasonic diagnostic apparatus system according to Embodiment 5.

FIG. 6 is a configuration explanatory view showing an ultrasonic diagnostic apparatus system according to Embodiment 6.

FIG. 7 is a configuration explanatory view showing an ultrasonic diagnostic apparatus system according to Embodiment 7.

FIG. 8 is a configuration explanatory view showing an ultrasonic diagnostic apparatus system according to Embodiment 8.

FIG. 9 is a configuration explanatory view showing an ultrasonic diagnostic apparatus system according to Embodiment 9.

FIG. 10 is a configuration explanatory view showing an ultrasonic diagnostic apparatus system according to Embodiment 10.

FIG. 11 is a configuration explanatory view showing an ultrasonic diagnostic apparatus system according to Embodiment 11.

FIG. 12 is a configuration explanatory view showing an ultrasonic diagnostic apparatus according to Embodiment 12.

### DETAILED DESCRIPTION OF THE INVENTION

The invention will be described below in more detail with embodiments shown in drawings. This does not limit the invention.

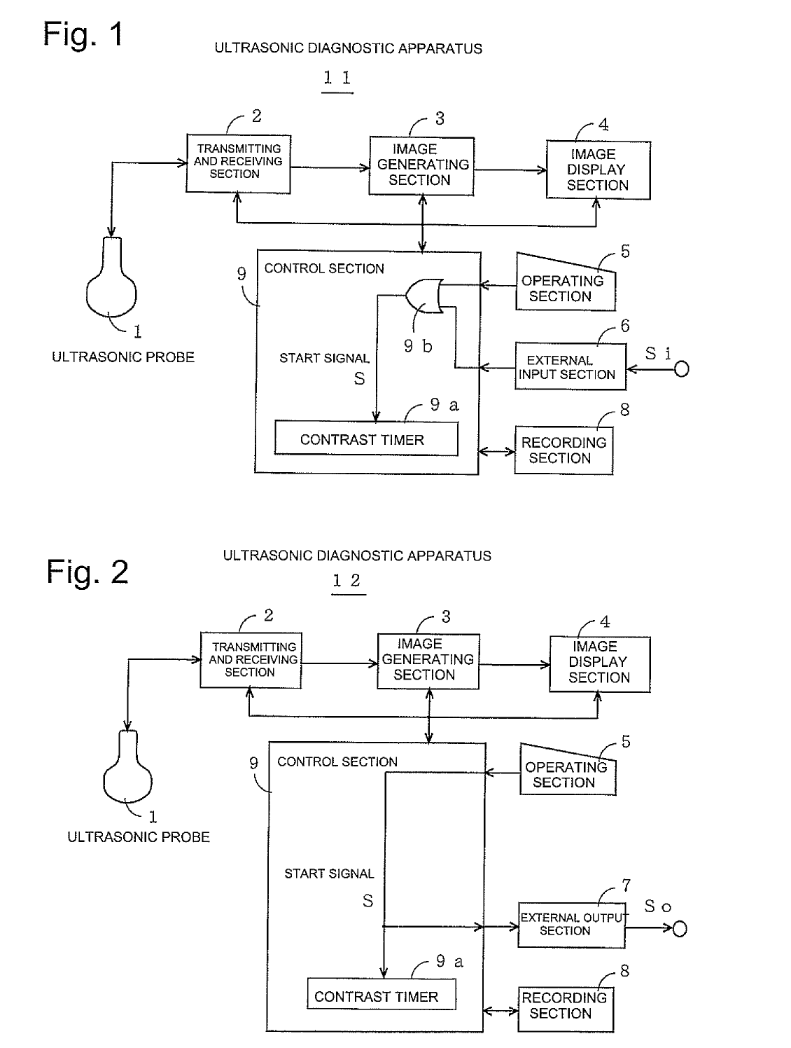

Embodiment 1. FIG. 1 is a configuration explanatory view of an ultrasonic diagnostic apparatus 11 according to Embodiment 1.

The ultrasonic diagnostic apparatus 11 includes an ultrasonic probe 1, a transmitting and receiving section 2 for driving the ultrasonic probe 1 to scan the inside of a subject with ultrasonic beams, an image generating section 3 for generating an ultrasonic image, an image display section 4 for displaying the ultrasonic image, an operating section 5 for use by an operator to provide an instruction and data for producing a start signal (hereinafter, simply called a start signal) S of a contrast timer, an external input section 6 for externally inputting the start signal Sᵢ, a recording section 8 for recording the ultrasonic image, and a control section 9 for controlling the whole.

The control section 9 includes a contrast timer 9a, and an OR circuit 9b inputting the start signal S produced by operation of the operating section 5 and the externally inputted start signal S to the contrast timer 9a.

Embodiment 2. FIG. 2 is a configuration explanatory view of an ultrasonic diagnostic apparatus 12 according to Embodiment 2.

The ultrasonic diagnostic apparatus 12 includes the ultrasonic probe 1, the transmitting and receiving section 2 for driving the ultrasonic probe 1 to scan the inside of a subject with ultrasonic beams, the image generating section 3 for generating the ultrasonic image, the image display section 4 for displaying the ultrasonic image, the operating section 5 for use by an operator to provide an instruction and data for producing the start signal Sₒ, an external output section 7 for externally outputting the start signal Sᵢ, the recording section 8 for recording the ultrasonic image, and the control section 9 for controlling the whole.

The control section 9 includes the contrast timer 9a.

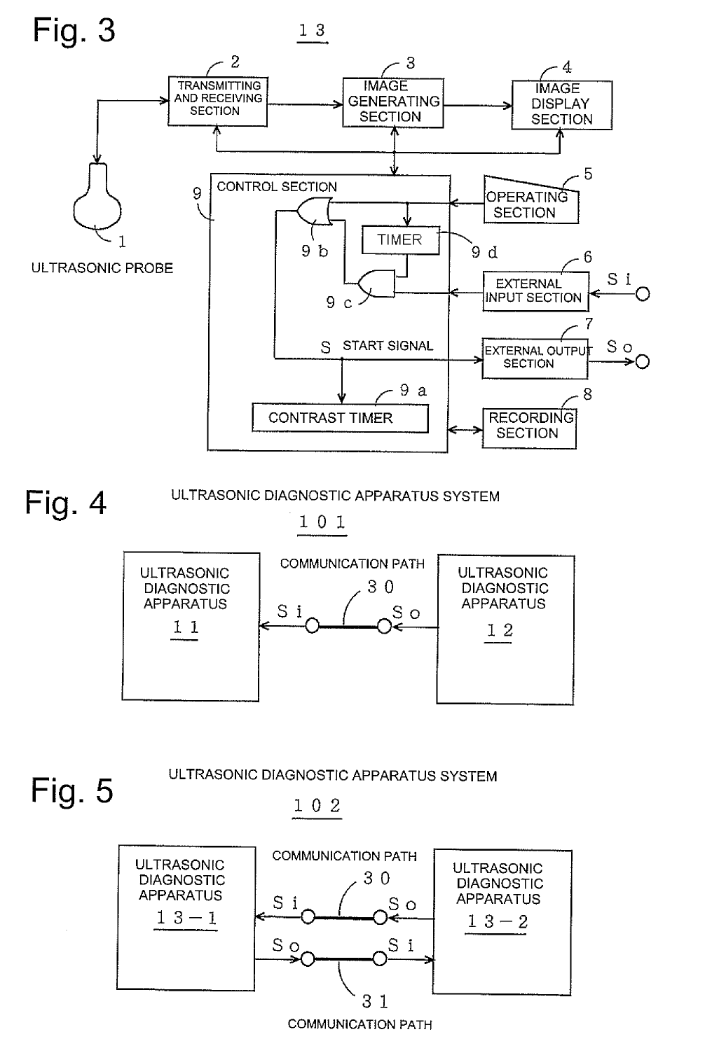

Embodiment 3. FIG. 3 is a configuration explanatory view of an ultrasonic diagnostic apparatus 13 according to Embodiment 3.

The ultrasonic diagnostic apparatus 13 includes the ultrasonic probe 1, the transmitting and receiving section 2 for driving the ultrasonic probe 1 to scan the inside of a subject with ultrasonic beams, the image generating section 3 for generating the ultrasonic image, the image display section 4 for displaying the ultrasonic image, the operating section 5 for use by an operator to provide an instruction and data for producing the start signal S, the external input section 6 for externally inputting the start signal Sᵢ, the external output section 7 for externally outputting the start signal Sₒ, the recording section 8 for recording the ultrasonic image, and the control section 9 for controlling the whole.

The control section 9 includes the contrast timer 9a, a gate circuit 9c controlling whether the externally inputted start signal S is passed therethrough or not, the OR circuit 9b inputting the start signal S produced by operation of the operating section 5 and the start signal S passed through the gate circuit 9c to the contrast timer 9a, and a timer circuit 9d which does not pass the externally inputted start signal through the gate circuit 9c for a predetermined time after the start signal S is produced by operation of the operating section 5.

Embodiment 4. FIG. 4 is a configuration explanatory view of an ultrasonic diagnostic apparatus system 101 according to Embodiment 4.

The ultrasonic diagnostic apparatus system 101 connects the external input section 6 of the ultrasonic diagnostic apparatus 11 according to Embodiment 1 and the external output section 7 of the ultrasonic diagnostic apparatus 12 according to Embodiment 2 by a (wired or wireless) communication path 30.

In the ultrasonic diagnostic apparatus system 101, the start signal S is produced by operation of the operating section 5 of the ultrasonic diagnostic apparatus 12. Accordingly, the contrast timers 9a of the ultrasonic diagnostic apparatus 11 and the ultrasonic diagnostic apparatus 12 can be started at the same time.

Embodiment 5. FIG. 5 is a configuration explanatory view of an ultrasonic diagnostic apparatus system 102 according to Embodiment 5.

The ultrasonic diagnostic apparatus system 102 connects the external input section 6 of a first ultrasonic diagnostic apparatus 13-1 according to Embodiment 3 and the external output section 7 of a second ultrasonic diagnostic apparatus 13-2 according to Embodiment 3 by the communication path 30, and connects the external output section 7 of the first ultrasonic diagnostic apparatus 13-1 and the external input section 6 of the second ultrasonic diagnostic apparatus 13-2 by a communication path 31.

In the ultrasonic diagnostic apparatus system 102, the start signal S is produced by operation of the operating section 5 of the ultrasonic diagnostic apparatus 13-1 or 13-2. Accordingly, the contrast timers 9a of the ultrasonic diagnostic apparatuses 13-1 and 13-2 can be started at the same time. The gate circuit 9c and the timer 9d can prevent the start signal S from being circulated in a loop.

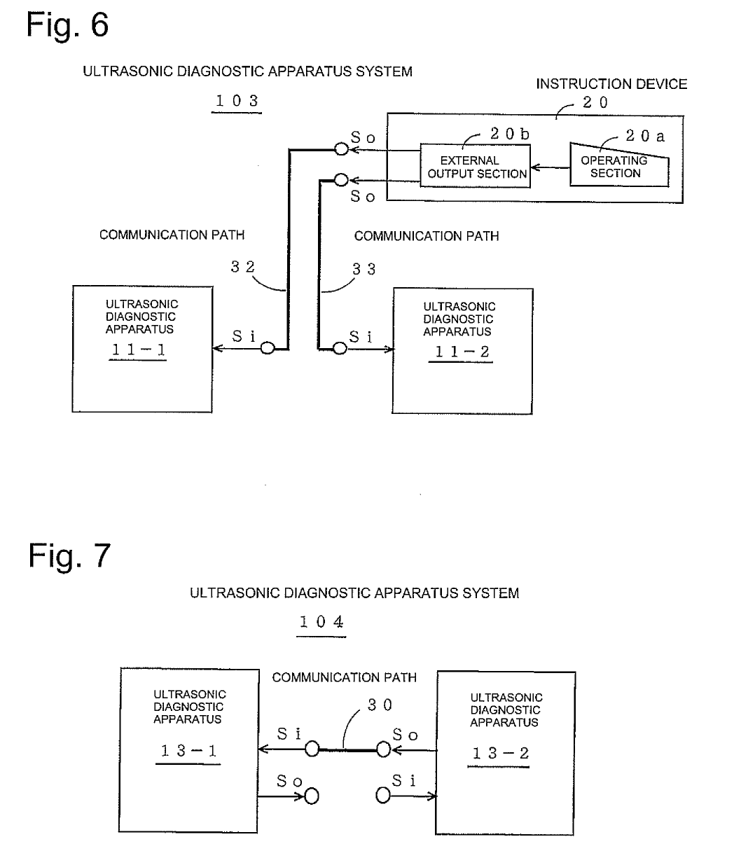

Embodiment 6. FIG. 6 is a configuration explanatory view of an ultrasonic diagnostic apparatus system 103 according to Embodiment 6.

The ultrasonic diagnostic apparatus system 103 includes an instruction device 20.

The instruction device 20 includes an operating section 20a for use by an operator to provide an instruction for producing the start signal S and an external output section 20b for externally outputting a plurality of the start signals Sₒ. The instruction device 20 connects the external input section 6 of a first ultrasonic diagnostic apparatus 11-1 according to Embodiment 1 and the external output section 20b of the instruction device 20 by a communication path 32, and connects the external input section 6 of a second ultrasonic diagnostic apparatus 11-2 according to Embodiment 1 and the external output section 20b of the instruction device 20 by a communication path 33.

In the ultrasonic diagnostic apparatus system 103, the start signal S is produced by operation of the operating section 20a of the instruction device 20. Accordingly, the contrast timers 9a of the ultrasonic diagnostic apparatuses 11-1 and 11-2 can be started at the same time.

Embodiment 7. FIG. 7 is a configuration explanatory view of an ultrasonic diagnostic apparatus system 104 according to Embodiment 7.

The ultrasonic diagnostic apparatus system 104 connects the external input section 6 of the first ultrasonic diagnostic apparatus 13-1 according to Embodiment 3 and the external output section 7 of the second ultrasonic diagnostic apparatus 13-2 according to Embodiment 3 by the communication path 30.

In the ultrasonic diagnostic apparatus system 104, the start signal S is produced by operation of the operating section 5 of the ultrasonic diagnostic apparatus 13-2. Accordingly, the contrast timers 9a of the ultrasonic diagnostic apparatuses 13-1 and 13-2 can be started at the same time.

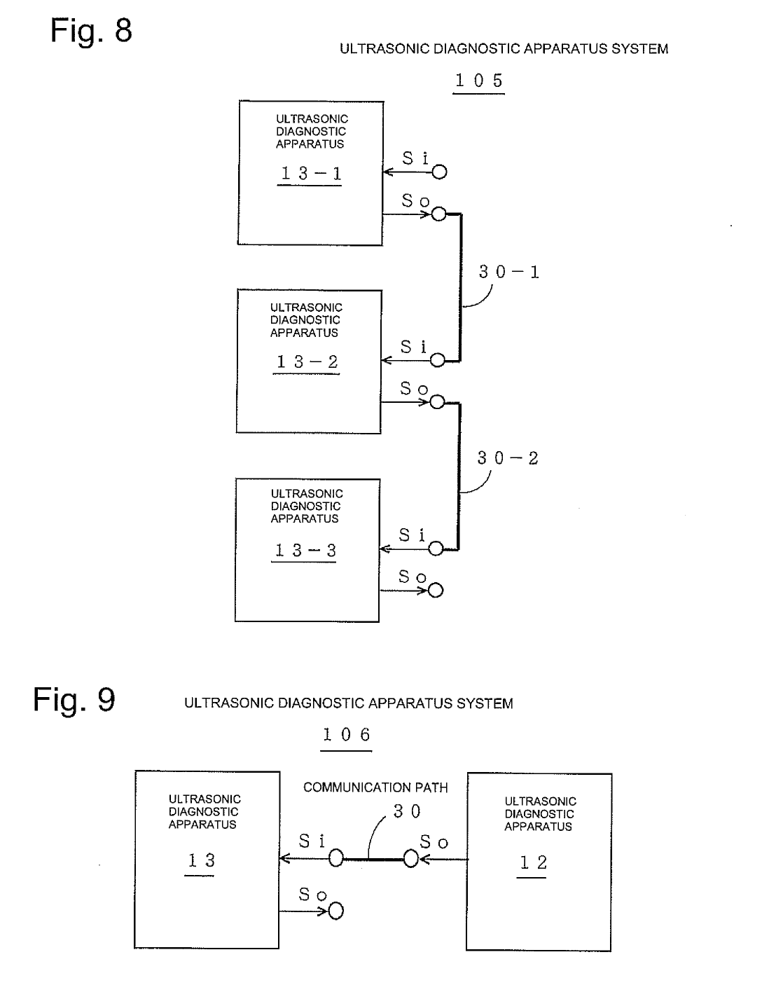

Embodiment 8. FIG. 8 is a configuration explanatory view of an ultrasonic diagnostic apparatus system 105 according to Embodiment 8.

The ultrasonic diagnostic apparatus system 105 connects the external output section 7 of the first ultrasonic diagnostic apparatus 13-1 according to Embodiment 3 and the external input section 6 of the second ultrasonic diagnostic apparatus 13-2 according to Embodiment 3 by a communication path 30-1, and connects the external output section 7 of the second ultrasonic diagnostic apparatus 13-2 and the external input section 6 of a third ultrasonic diagnostic apparatus 13-3 according to Embodiment 3 by a communication path 30-2.

In the ultrasonic diagnostic apparatus system 105, the start signal S is produced by operation of the operating section 5 of the ultrasonic diagnostic apparatus 13-1. Accordingly, the contrast timers 9a of the ultrasonic diagnostic apparatuses 13-1, 13-2, and 13-3 can be started at the same time.

Embodiment 9. FIG. 9 is a configuration explanatory view of an ultrasonic diagnostic apparatus system 106 according to Embodiment 9.

The ultrasonic diagnostic apparatus system 106 connects the external input section 6 of the ultrasonic diagnostic apparatus 13 according to Embodiment 3 and the external output section 7 of the ultrasonic diagnostic apparatus 12 according to Embodiment 2 by the communication path 30.

In the ultrasonic diagnostic apparatus system 106, the start signal S is produced by operation of the operating section 5 of the ultrasonic diagnostic apparatus 12. Accordingly, the contrast timers 9a of the ultrasonic diagnostic apparatuses 11 and 13 can be started at the same time.

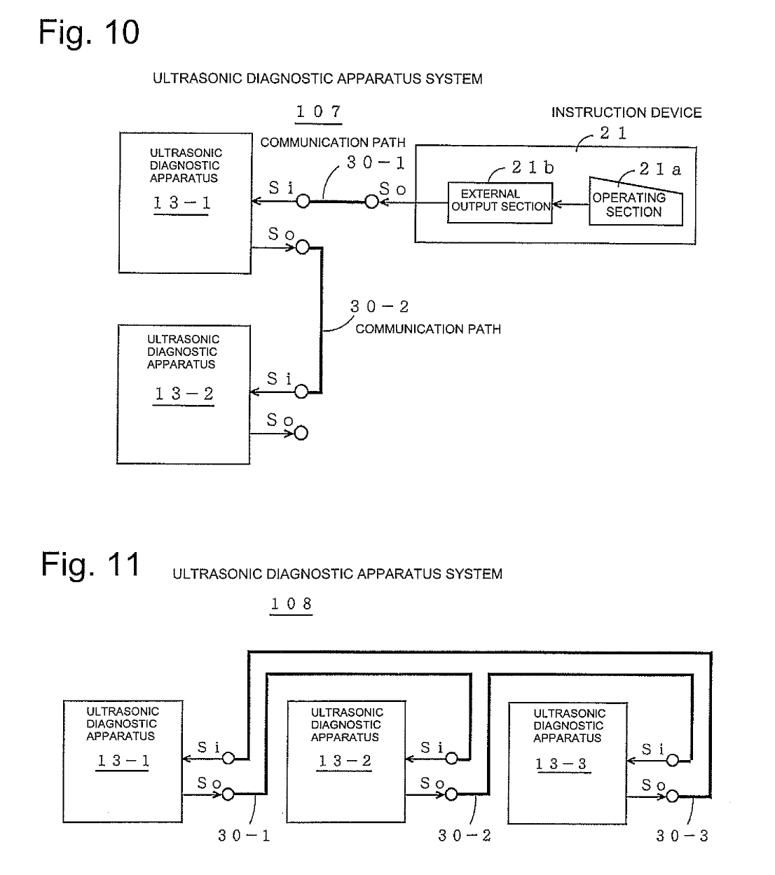

Embodiment 10. FIG. 10 is a configuration explanatory view of an ultrasonic diagnostic apparatus system 107 according to Embodiment 10.

The ultrasonic diagnostic apparatus system 107 includes an instruction device 21.

The instruction device 21 includes an operating section 21a for use by an operator to provide an instruction for producing the start signal S and an external output section 21b for externally outputting the start signal Sₒ. The instruction device 21 connects the external input section 6 of the first ultrasonic diagnostic apparatus 13-1 according to Embodiment 3 and the external output section 21b of the instruction device 21 by the communication path 30-1, and connects the external input section 6 of the second ultrasonic diagnostic apparatus 13-2 according to Embodiment 3 and the external output section 7 of the first ultrasonic diagnostic apparatus 13-1 by the communication path 30-2.

In the ultrasonic diagnostic apparatus system 107, the start signal S is produced by operation of the operating section 21a of the instruction device 21. Accordingly, the contrast timers 9a of the ultrasonic diagnostic apparatuses 13-1 and 13-2 can be started at the same time.

Embodiment 11. FIG. 11 is a configuration explanatory view of an ultrasonic diagnostic apparatus system 108 according to Embodiment 11.

The ultrasonic diagnostic apparatus system 108 connects the external output section 7 of the first ultrasonic diagnostic apparatus 13-1 according to Embodiment 3 and the external input section 6 of the second ultrasonic diagnostic apparatus 13-2 according to Embodiment 3 by the communication path 30-1, connects the external output section 7 of the second ultrasonic diagnostic apparatus 13-2 and the external input section 6 of the third ultrasonic diagnostic apparatus 13-3 according to Embodiment 3 by the communication path 30-2, and connects the external output section 7 of the third ultrasonic diagnostic apparatus 13-3 and the external input section 6 of the first ultrasonic diagnostic apparatus 13-1 by a communication path 30-3.

In the ultrasonic diagnostic apparatus system 108, the start signal S is produced by operation of the operating section 5 of any one of the ultrasonic diagnostic apparatuses 13-1, 13-2, and 13-3. Accordingly, the contrast timers 9a of the ultrasonic diagnostic apparatuses 13-1, 13-2, and 13-3 can be started at the same time. The gate circuit 9c and the timer 9d of the ultrasonic diagnostic apparatus (any one of 13-1, 13-2, and 13-3) producing the start signal S by operation of the operating section 5 can prevent the start signal S from being circulated in a loop.

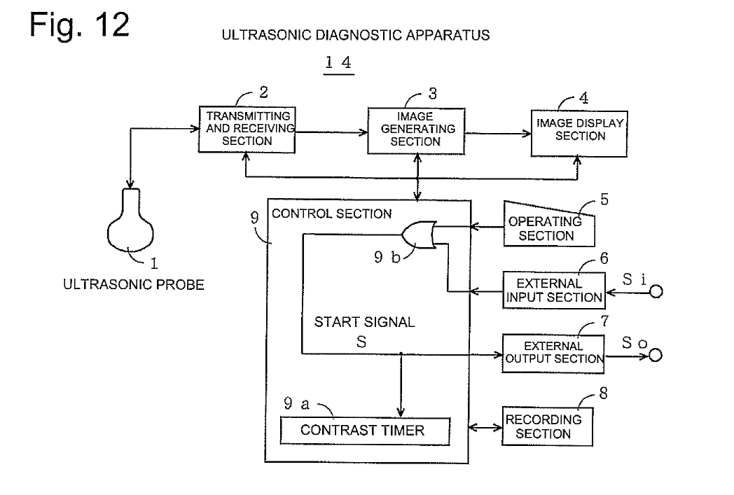

Embodiment 12. FIG. 12 is a configuration explanatory view of an ultrasonic diagnostic apparatus 14 according to Embodiment 12.

The ultrasonic diagnostic apparatus 14 includes the ultrasonic probe 1, the transmitting and receiving section 2 for driving the ultrasonic probe 1 to scan the inside of a subject with ultrasonic beams, the image generating section 3 for generating an ultrasonic image, an image display section 4 for displaying the ultrasonic image, the operating section 5 for use by an operator to provide an instruction and data for producing the start signal S, the external input section 6 for externally inputting the start signal Sᵢ, the external output section 7 for externally outputting the start signal Sₒ, the recording section 8 for recording the ultrasonic image, and the control section 9 for controlling the whole.

The control section 9 includes the contrast timer 9a and the OR circuit 9b inputting the start signal S produced by operation of the operating section 5 and the externally inputted start signal S to the contrast timer 9a.

The ultrasonic diagnostic apparatus 14 which is not connected in a loop can configure the same ultrasonic diagnostic apparatus system as the ultrasonic diagnostic apparatus 13 according to Embodiment 3.

Embodiment 13. In Embodiments 1 to 12, only the start signal S starting time measurement of the contrast timer 9a is described. The invention can be applied likewise for a stop signal (or a freeze signal) stopping time measurement of the contrast timer 9a and a reset signal returning the time measured value of the contrast timer 9a to “0”. In this case, the contrast timers of a plurality of ultrasonic diagnostic apparatuses can be started/stopped/reset at the same time.

Many widely different embodiments of the invention may be configured without departing from the spirit and the scope of the present invention. It should be understood that the present invention is not limited to the specific embodiments described in the specification, except as defined in the appended claims.

1. An ultrasonic diagnostic apparatus comprising:
a contrast timer starting time measurement by a start signal; and
an external input device for externally inputting said start signal.

2. An ultrasonic diagnostic apparatus comprising:
a contrast timer starting time measurement by a start signal;
an operating device for use by an operator to perform operation for producing said start signal; and
an external output device for externally outputting said start signal.

3. An ultrasonic diagnostic apparatus according to claim 1, further comprising:
an operating device for use by an operator to perform operation for producing said start signal; and
an external output device for externally outputting said start signal.

4. The ultrasonic diagnostic apparatus according to claim 3, farther comprising:
an external input inhibiting device for inhibiting an external input of the start signal for a predetermined time after the start signal is produced by operation of said operating device.

5. An ultrasonic diagnostic apparatus system comprising:
a first ultrasonic diagnostic apparatus comprising:
a contrast timer starting time measurement by a start signal; and
an external input device for externally inputting said start signal;
a second ultrasonic diagnostic apparatus comprising:
a contrast timer starting time measurement by a start signal;
an operating device for use by an operator to perform operation for producing said start signal; and
an external output device for externally outputting said start signal; and
a communicating device for connecting the external input device of said first ultrasonic diagnostic apparatus and the external output device of said second ultrasonic diagnostic apparatus.

6. An ultrasonic diagnostic apparatus system comprising:
a first and a second ultrasonic diagnostic apparatus each of said first and second ultrasonic diagnostic apparatus comprising:
a contrast timer starting time measurement by a start signal;
an external input device for externally inputting said start signal:.
an operating device for use by an operator to perform operation for producing said start signal;
an external output device for externally outputting said start signal; and
an external input inhibiting device for inhibiting an external input of the start signal for a predetermined time after the start signal is produced by operation of said operating device;
a first communicating device for connecting the external output device of said first ultrasonic diagnostic apparatus and the external input device of said second ultrasonic diagnostic apparatus; and
a second communicating device for connecting the external input device of said first ultrasonic diagnostic apparatus and the external output device of said second ultrasonic diagnostic apparatus.

7. An ultrasonic diagnostic apparatus system comprising:
a first and a second ultrasonic diagnostic each of said first and second ultrasonic diagnostic apparatus comprising:
a contrast timer starting time measurement by a start signal; and
an external input device for externally inputting said start signal;
an instruction device having an operating device for use by an operator to perform operation for producing a start signal and an external output device for externally outputting a plurality of said start signals; and
a communicating device for connecting the external input device of said first ultrasonic diagnostic apparatus, the external input device of said second ultrasonic diagnostic apparatus, and the external output device of said instruction device.

8. An ultrasonic diagnostic apparatus system comprising:
a first and a second ultrasonic diagnostic apparatus each of said first and second ultrasonic diagnostic apparatus comprising:
a contrast timer starting time measurement by a start signal;
an external input device for externally inputting said start signal;
an operating device for use by an operator to perform operation for producing said start signal; and
an external output device for externally outputting said start signal; and
a communicating device for connecting the external output device of said first ultrasonic diagnostic apparatus and the external input device of said second ultrasonic diagnostic apparatus.

9. An ultrasonic diagnostic apparatus system comprising:
a first and a second ultrasonic diagnostic apparatus each of said first and second ultrasonic diagnostic apparatus comprising:
a contrast timer starting time measurement by a start signal,
an external input device for externally inputting said start signal,
an operating device for use by an operator to perform operation for producing said start signal:
an external output device for externally outputting said start signal; and
an external input inhibiting device for inhibiting an external input of the start signal for a predetermined time after the start signal is produced by operation of said operating device, and
a communicating device for connecting the external output device of said first ultrasonic diagnostic apparatus and the external input device of said second ultrasonic diagnostic apparatus.

10. An ultrasonic diagnostic apparatus system comprising, when n=1, 2, . . . , N−1, N≧3:
first to N-th ultrasonic diagnostic apparatuses comprising:
a contrast timer starting time measurement by a start signal,
an external input device for externally inputting said start signal;
an operating device for use by an operator to, perform operation for producing said start signal, and
an external output device for externally outputting said start signal; and
an n-th communicating device for connecting the external output device of said n-th ultrasonic diagnostic apparatus and the external input device of said (n+1)-th ultrasonic diagnostic apparatus.

11. An ultrasonic diagnostic apparatus system comprising, when n=1, 2, . . . , N−1,N≧3:
first to N-th ultrasonic diagnostic apparatuses and comprising:
a contrast timer starting time measurement by a start signal,
an external input device for externally inputting said start signal:
an operating device for use by an operator to perform operation for producing said start signal;
an external output device for externally outputting said start signal; and
an external input inhibiting device for inhibiting an external input of the start signal for a predetermined time after the start signal is produced by operation of said operating device; and
an n-th communicating device for connecting the external output device of said n-th ultrasonic diagnostic apparatus and the external input device of said (n+1)-th ultrasonic diagnostic apparatus.

12. An ultrasonic diagnostic apparatus system comprising:
a first ultrasonic diagnostic apparatus comprising:
a contrast timer starting time measurement by a start signal;
an operating device for use by an operator to perform operation for producing said start signal; and
an external output device for externally outputting said start signal;
a second ultrasonic diagnostic apparatus comprising:
a contrast timer starting time measurement by a start signal;
an external input device for externally inputting said start signal;
an operating device for use by an operator to perform operation for producing said start signal; and
an external output device for externally outputting said start signal; and
a communicating device for connecting the external output device of said first ultrasonic diagnostic and the external input device of said second ultrasonic diagnostic apparatus.

13. An ultrasonic diagnostic apparatus system comprising:
a first ultrasonic diagnostic apparatus comprising:
a contrast timer starting time measurement by a start signal;
an operating device for use by an operator to perform operation for producing said start signal; and
an external output device for externally outputting said start signal;
a second ultrasonic diagnostic apparatus comprising:
a contrast timer starting time measurement by a start signal;
an external input device for externally inputting said start signal;
an operating device for use by an operator to perform operation for producing said start signal;
an external output device for externally outputting said start signal; and
an external input inhibiting device for inhibiting an external input of the start signal for a predetermined time after the start signal is produced by operation of said operating device; and
a communicating device for connecting the external output device of said first ultrasonic diagnostic apparatus and the external input device of said second ultrasonic diagnostic apparatus.

14. An ultrasonic diagnostic apparatus system comprising:
a first and a second ultrasonic diagnostic apparatus each of said first and second ultrasonic diagnostic apparatus comprising;
a contrast timer starting time measurement by a start signal;
an external input device for externally inputting said start signal
an operating device for use by an operator to perform operation for producing said start signal; and
an external output device for externally outputting said start signal;
an instruction device having an operating device for use by an operator to perform operation for producing a start signal and an external output device for externally outputting said start signal;
a first communicating device for connecting the external input device of said first ultrasonic diagnostic apparatus and the external output device of said instruction device; and
a second communicating device for connecting the external output device of said first ultrasonic diagnostic apparatus and the external input device of said second ultrasonic diagnostic apparatus.

15. An ultrasonic diagnostic apparatus system comprising:
a first and a second ultrasonic diagnostic apparatus each of said first and second ultrasonic diagnostic apparatus comprising:
a contrast timer starting time measurement by a start signal;
an external input device for externally inputting said start signal;
an operating device for use by an operator to perform operation for producing said start signal;
an external output device for externally outputting said start signal; and
an external input inhibiting device for inhibiting an external input of the start signal for a predetermined time after the start signal is produced by operation of said operating device;
an instruction device having an operating device for use by an operator to perform operation for producing a start signal and an external output device for externally outputting said start signal;
a first communicating device for connecting the external input device of said first ultrasonic diagnostic apparatus and the external output device of said instruction device; and
a second communicating device for connecting the external output device of said first ultrasonic diagnostic apparatus and the external input device of said second ultrasonic diagnostic apparatus.

16. An ultrasonic diagnostic apparatus system comprising, when n=1, 2, . . . , N−1, N≧3:
first to N-th ultrasonic diagnostic apparatuses; comprising:
a contrast timer starting time measurement by a start signal;
an external input device for externally inputting said start signal;
an operating device for use by an operator to perform operation for producing said start signal;
an external output device for externally outputting said start signal; and
an external input inhibiting device for inhibiting an external input of the start signal for a predetermined time after the start signal is produced by operation of said operating device;
an n-th communicating device for connecting external output device of said n-th ultrasonic diagnostic apparatus and the external input device of said (n+1)-th ultrasonic diagnostic apparatus; and
an N-th communicating device for connecting the external output device of said N-th ultrasonic diagnostic apparatus and the external input device of said first ultrasonic diagnostic apparatus.

17. An ultrasonic diagnostic apparatus comprising:
a contrast timer starting time measurement by a start signal and stopping time measurement by a stop signal;
an operating device for use by an operator to perform operation for producing said start signal and said stop signal;
an external output device for externally outputting said start signal and said stop signal; and
an external input device for externally inputting said start signal and said stop signal.

18. An ultrasonic diagnostic apparatus system comprising:
a first and a second ultrasonic diagnostic apparatus each of said first and second ultrasonic diagnostic apparatus comprising:
a contrast timer starting time measurement by a start signal and stopping time measurement by a stop signal;
an operating device for use by an operator to perform operation for producing said start signal and said stop signal;
an external output device for externally outputting said start signal and said stop signal; and
an external input device for externally inputting said start signal and said stop signal, and
a communicating device for connecting the external output device of said first ultrasonic diagnostic apparatus and the external input device of said second ultrasonic diagnostic apparatus.

19. An ultrasonic diagnostic apparatus system comprising, when n=1, 2, . . . , N−1, N≧3:
first to N-th ultrasonic diagnostic apparatuses comprising:
a contrast timer starting time measurement by a start signal; and
an external input device for externally inputting said start signal; and
an n-th communicating device for connecting the external output device of said n-th ultrasonic diagnostic apparatus and the external input device of said (n+1)-th ultrasonic diagnostic apparatus.
